(19) United States
(12) Patent Application Publication
(10) Pub. No.: US 2012/0172677 A1
(43) Pub. Date: Jul. 5, 2012

(54) SYSTEMS AND METHODS FOR MONITORING AND PROCESSING BIOMETRIC DATA

(76) Inventors: Robert J. Logan, Poway, CA (US); Dana C. Dorroh, Raleigh, NC (US); Thomas M. Gordon, III, Raleigh, NC (US); Barry H. Beith, Cary, NC (US)

(21) Appl. No.: 13/335,919

(22) Filed: Dec. 22, 2011

Related U.S. Application Data
(60) Provisional application No. 61/428,845, filed on Dec. 30, 2010.

(51) Int. Cl. A61B 5/01; A61B 5/08; A61B 5/1455; A61B 5/103; A61B 5/087

(52) U.S. Cl. 600/301

(57) ABSTRACT

Methods and systems for monitoring biometric and/or environmental data of a user are provided. The user monitoring system includes a dental appliance configured to fit substantially inside a mouth of a user, a temperature sensor for measuring user temperature, a pressure sensor for measuring pressure exerted by the user, and a processor for acquiring and processing temperature and pressure data of the user. The user monitoring system may communicate with an observer monitoring system. In some embodiments, the user monitoring system also measures one or more of blood oxygen saturation level, proper placement of the dental appliance, ambient air temperature, light levels and respiration rate.

### CROSS REFERENCE TO RELATED APPLICATIONS

This non-provisional application claims the benefit of provisional application No. 61/428,845, filed Dec. 30, 2010, which application is incorporated herein in its entirety by this reference.

This application is also related to an application being filed concurrently herewith, application Ser. No. ______, by Robert J. Logan et al., entitled “Systems and Methods for Monitoring and Processing Biometric Data,” (Our Ref: HCP-1102), also incorporated herein in its entirety by this reference.

Additionally, this application is also related to an application being filed concurrently herewith, application Ser. No. ______, by Robert J. Logan et al., entitled “Systems and Methods for Monitoring and Processing Biometric Data,” (Our Ref: HCP-1103), also incorporated herein in its entirety by this reference.

### BACKGROUND

The present invention relates to systems and methods for monitoring a variety of biometric data and environmental data primarily via a dental appliance worn by a human user.

U.S. Pat. No. 7,481,773 discloses a body temperature monitoring system which includes a mouth guard, a temperature-sensing unit associated with the mouth guard, and an indicator unit responsive to the temperature sensing unit. The indicator unit indicates if a body temperature sensed by the temperature-sensing unit is outside of a pre-selected range. The indicator unit may be programmed to actuate an indicator when the temperature-sensing unit senses one or more temperatures that fall outside the pre-selected range. In one implementation, the indicator unit receives a string of multiple temperature readings and determines which temperatures are valid and invalid, and averages the valid temperatures. A method of monitoring a person's body temperature that parallels the above device is also disclosed. The '773 patent also discloses numerous ways to monitor temperature data to users of the device through mechanical (wireless or wired), audio, visual, and physical means.

It is therefore apparent that an urgent need exists for an improved real-time monitoring system capable of monitoring a variety of biometric data of one or more users and environmental in their respective environments. This improved monitoring system increases individual safety and performance before, during, and after the monitored session.

The monitoring system is embodied in an oral appliance that can be made with one or more materials, and be placed in an individual's mouth in a variety of positions, locations, and form factors.

### SUMMARY

To achieve the foregoing and in accordance with the present invention, systems and methods for monitoring biometric and environmental parameters are provided.

For example, these monitoring systems and methods enable an individual user and/or an observer, such as a coach, trainer, supervisor, or guardian, to closely monitor one or more users in real time or post event. The objective of the monitoring is to enhance individuals' safety and performance in a variety of situations including in athletics, workplace, home, military, firefighting, and recreation.

In one embodiment, the computerized user monitoring system includes a dental appliance configured to fit substantially inside a mouth of a user. The dental appliance includes one or more temperature sensors. The dental appliance may include one or more additional sensors, such as force, pressure, and/or biometric sensors. The dental appliance also includes a processor coupled to the temperature sensors and any other sensors. The monitoring system may also include one or more display technologies including visual, tactile, and/or audible indicators coupled to the processor.

In some embodiments, the user monitoring system includes a transmitter for sending data to an external monitoring system worn by the user, so that the user can track their own health and/or performance and be alerted under a wide variety of conditions in real-time, thereby enabling the individual to take any appropriate action. This external user monitoring system includes data storage capabilities that enable the user to review, process, and conduct analyses of their data including milestones, trends, and physiological events to further enhance their well being, performance and fitness.

In some embodiments, the user monitoring system includes a transmitter for sending data to an external observer monitoring system, so that the observer can track the health and/or performance of one or more users and be alerted under a wide variety of conditions in real-time, enabling the observer to take any appropriate action. This external observer monitoring system includes a data storage capability that allows the observer to review and to further assess the users' data for future review, processing, analysis and trends.

In some embodiments, the user monitoring system includes a transceiver for sending information to the observer monitoring system and/or sending information from the observer monitoring system to the user monitoring system.

In some embodiments, the user monitoring system includes chambers for capturing fluids such as saliva and/or air samples in the dental appliance. In some cases, the additional materials may be added to the oral appliance to facilitate the collection and storage of the fluid and air samples. The collected samples may be analyzed locally in the dental appliances and/or remotely in a laboratory.

In some embodiments, the user monitoring system includes sensors that measure the amount of certain compounds including oxygen, carbon dioxide, and/or the presence of alcohol.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIG. 1 shows a perspective view illustrating one embodiment of the user monitoring system, in accordance with the present invention;

FIG. 2 shows an exploded view illustrating the components of the embodiment of FIG. 1;

FIG. 3 includes top, front, and side views of the embodiment of FIG. 1;

FIG. 4 shows an exploded view of another embodiment of the user monitoring system illustrating the components of the system of this embodiment in accordance with the present invention;

FIG. 5 is a perspective view of another embodiment of the user monitoring system in accordance with the present invention;

FIG. 6 shows an exploded view of another embodiment of the user monitoring system illustrating the components of the system of this embodiment in accordance with the present invention;

FIG. 7 is a diagram illustrating the customizable functions of the processing circuit in accordance with the present invention;

FIG. 8 illustrates the user monitoring system and observer monitoring system collecting and sharing biometric and environmental data via a two-way means of communication;

FIG. 9 illustrates the user monitoring system case in accordance with the present invention which improves form and fit for the user while helping to ensure ease of use in forming and storage;

FIG. 10 illustrates a summary of the present invention with the embodiment applicable to an athlete and related observer;

FIG. 11 illustrates the user monitoring system inductive charge case in accordance with the present invention. This is an inductively charged monitoring system;

FIG. 12 illustrates using kinetic energy from biting down on the appliance to generate electrical power for the appliance;

FIG. 13 shows another embodiment of the user monitoring system illustrating pulse oximeter technology in accordance with the present invention; and

FIG. 14 shows another embodiment of the user monitoring system illustrating capnograph technology in accordance with the present invention;

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

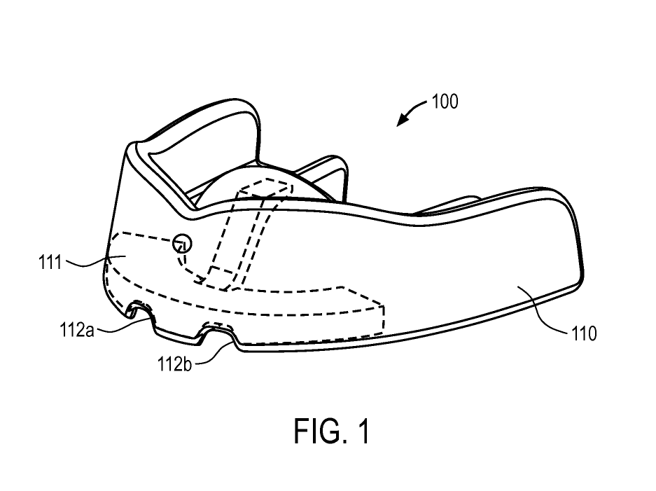

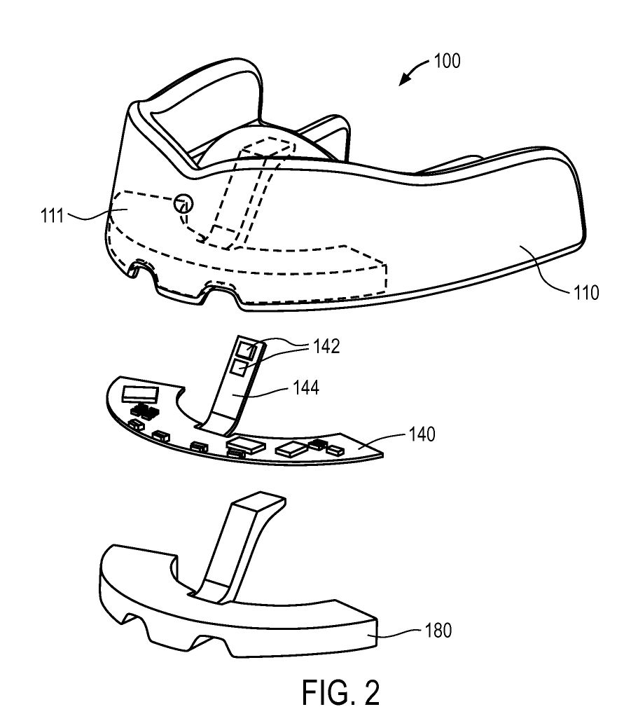

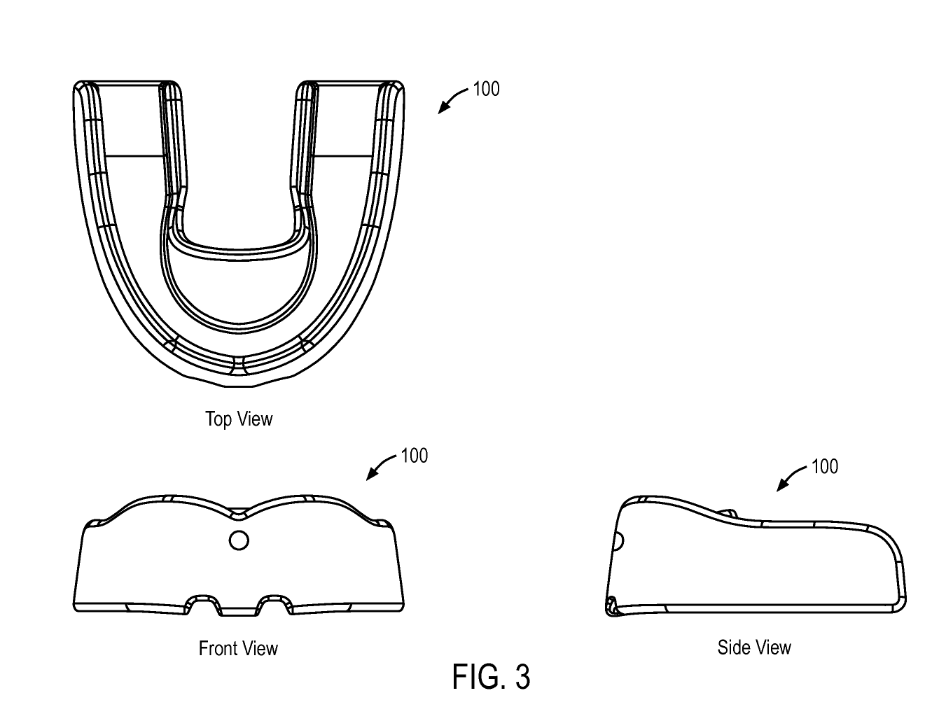

The present invention relates to systems and methods for monitoring a variety of biometric and environmental data related to one or more users at a variety of venues, such as workplaces and sporting or recreational facilities, enabling the user and/or one or more human observers, such as coaches, trainers, parents or supervisors, to monitor the health and performance of these users. To facilitate discussion, FIG. 1 is a perspective, showing one embodiment of the computerized monitoring system 100 in accordance to the present invention. FIG. 2 shows an exploded view illustrating the components of the embodiment of FIG. 1. FIG. 3 shows the top, front, and side views of the embodiment of FIG. 1.

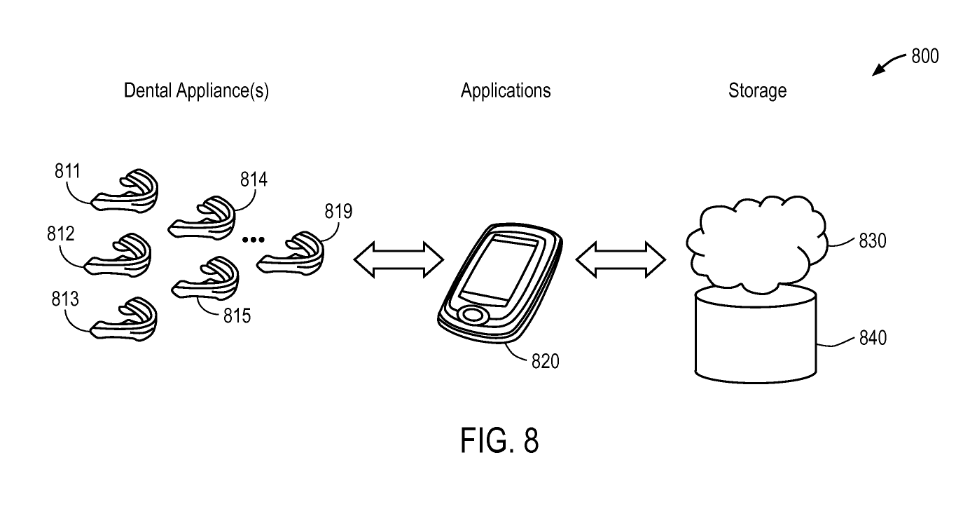

Referring also to FIG. 8 which illustrates an exemplary implementation 800 in accordance with an embodiment of the present invention, one or more (user) monitoring systems 811, 812 . . . 819 communicate with at least one (external) observer monitoring system, e.g. implemented into a smart phone 820, which in turn may be communicating with an optional storage system 840 via a computerized network 830, e.g., the Internet. Monitoring system 100 includes a dental appliance 110 with a recessed compartment 111 for housing a processor 140 which is shielded by a protective cover 180. The back-end repository not only houses and stores data, it also provides the vehicle and foundation for multiple analytics including, but not limited to, normative data, capturing individual data and tracking changes over time based on captured data from the monitoring system and combining that with other data like weather conditions and time of day. Data is aggregated over individual profiles based on body types, physical activities, and position of the individuals which can be combined with other individual's profiles to create a team profile or group profile of like individuals. Examples of dental appliance 110 include mouth guards, mouth pieces, dentures, dental flippers, braces, retainers, first responder breathing mouth pieces, ventilator mouth pieces, anti-snoring and teeth grinding mouth pieces, infant pacifier, and scuba or snorkeling mouth pieces. Accordingly, many variations (not all shown) of dental appliance 110 can be created for upper, lower, or upper and lower jaw configurations depending on the requirements of the user.

One or more sensors are strategically located on inside and/or on the surface of dental appliance 110. For example, sensors 142 can be located on an extension of a PCB 144 as shown on the embodiment of processor 140 of FIG. 2.

Dental appliance 110 can be made from a variety of materials that can vary in flexibility, weight and softness, depending on the intended use. It is also possible for the dental appliance 110 to be semi-customizable, for example, by using a heat-moldable material so that it can be heated to a semi-deformable state, and then placed into a specific user's mouth to semi-permanently conform to the particular user oral contour. Dental appliance 110 can be semi-customizable to include a “Boil & Bite” variety. It is also possible for dental appliance 110 to be fully customizable using molds made of the user's teeth and jaw. Alternate form factors of dental appliance 110 include a low profile device like an orthodontic retainer or similar devices made of a variety of materials including stainless steel, plastics and/or silicon.

The “Boil & Bite” process for dental appliance can be enhanced by pairing the dental appliance with a dental appliance storage case specifically designed for the requirements of a particular dental appliance design and material composition. The “Boil & Bite” process will aid the user's fit and comfort of the dental appliance.

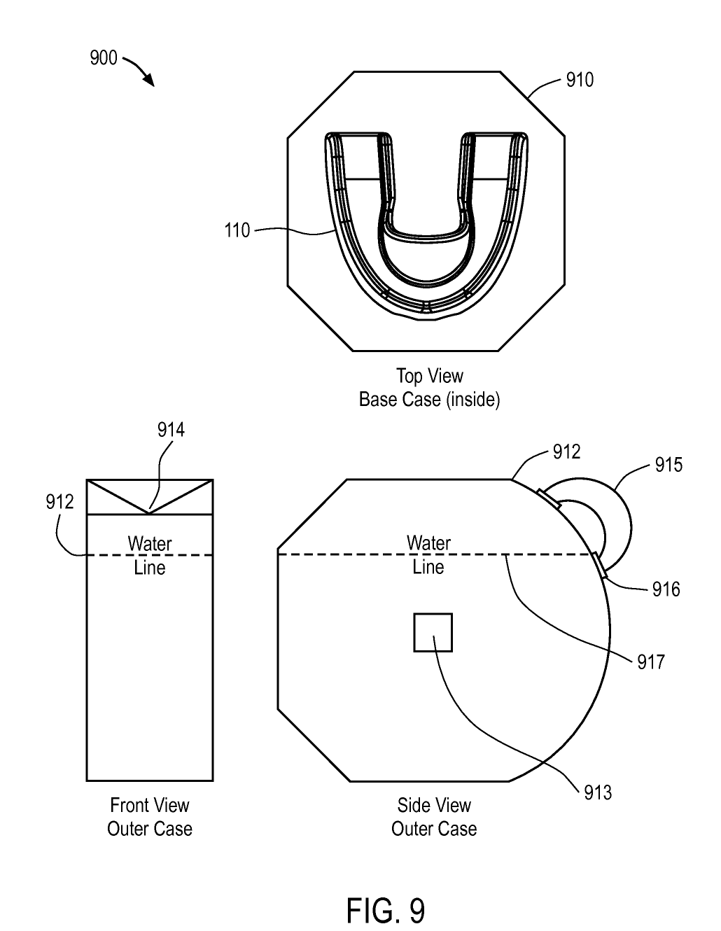

To facilitate further discussion, FIG. 9 shows the top, front, and side views of a dental appliance storage case 900 for the monitoring system that includes a base compartment 910 that fits into an outer compartment 912 which holds the base compartment. The dental appliance 110 sits in the base compartment 910, and base compartment 910 slides into the outer compartment 912. This storage case attaches through a coupler 913 to other storage cases in order to create a singular stack of storage cases for ease of transportation and ease of organization. This case 900 also serves as a platform for the implementation of the Boil & Bite embodiment described above. The benefits of case 900 includes providing the precise amount of boiling water, protection of the embedded electronic components inside dental appliance 110 while subjected to high heat, and providing a customized user fit. Note that water can be substituted with suitable alternative liquids, including chemical cleaning/disinfecting solutions, known to one skilled in the art.

The storage case 900 may include one or more the following components:
1. Base compartment 910 for storing dental appliance 110.
2. Outer compartment 912 for encasing the base compartment 910. This outer compartment 912 is visually marked with a “water line” 917 notation to provide the user with the proper amount of water to be boiled which gets poured onto the dental appliance 110. The outer compartment 912 also serves as a cooling tray for the dental appliance 110 to go from the boiling water to an ice bath after a predetermined period of time in the boiling water in the base compartment 910.
3. Pouring spout 914 on the outer compartment 912 which aids in the pouring of the water from the outer compartment 912 into the base compartment 910 onto the dental appliance.
4. Detachable, pouring handle 915 on the outer compartment which aids in the handling of the boiling water which is poured onto the dental appliance 110 as it sits in the base compartment 910.

Coupler 916 allows the detachable, pouring handle to be removed from the outer compartment 912 to then retrieve the dental appliance 110 from the base compartment 910 after appliance 110 has sat in the base compartment 910 with boiling water thereby further protecting the user from the heated water.

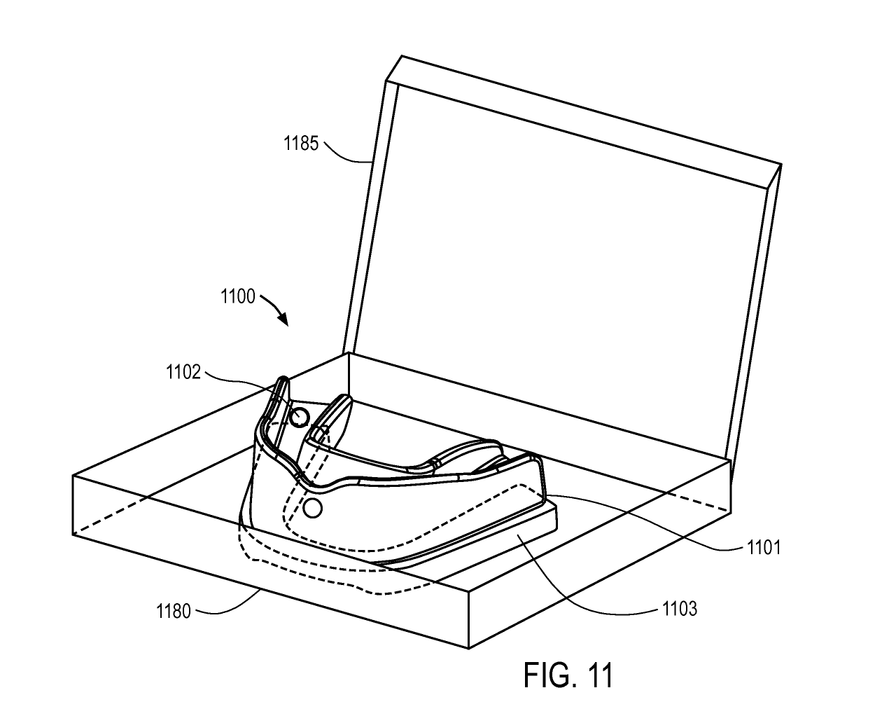

In some embodiments, dental appliance 1101 can be recharged using an inductive energy (power) transfer, as shown in FIG. 11. Inductive energy transfer is also known as wireless energy transfer or inductive power capture. The appliance 1101 would be able to be recharged from an inductive power source 1103 that uses an electromagnetic field to transmit power wirelessly from the power source 1103 to the appliance 1101. Hence, induction coils (not shown) of source 1103 are magnetically coupled to but do not touch the surface of the charger port 1102 of appliance 1101 when appliance 1101 sits on an inductive power source 1103 inside case 1180. This would enable appliance 1101 to provide body temperature measurements or to be recharged without exposing the electronics of appliance 1101 to outside contaminates.

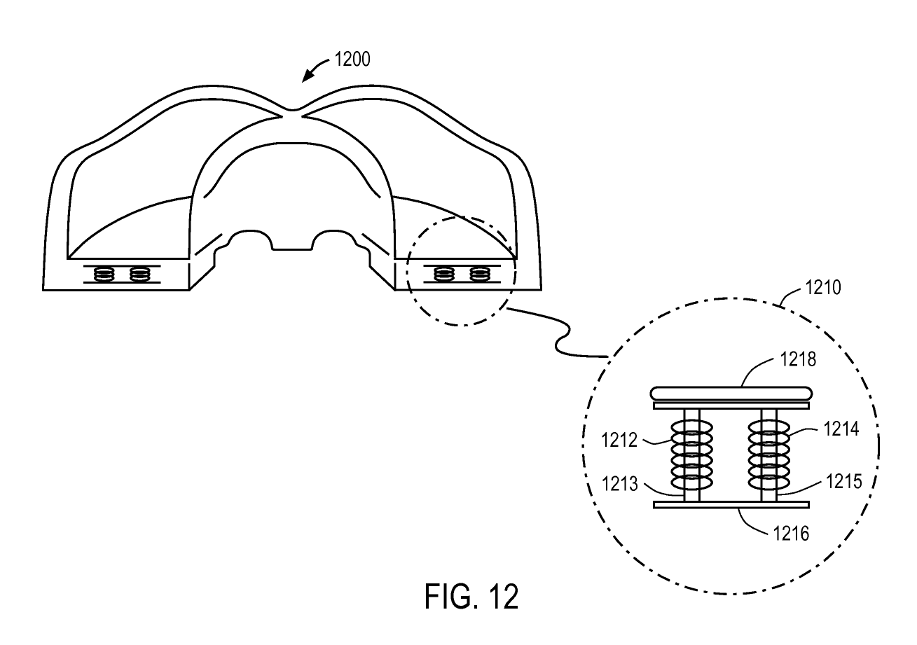

As shown in FIG. 12, in some embodiments, the dental appliance 1200 can be recharged by capturing and converting the energy generated by the user biting down on the appliance 1200 in a normal course of wearing the appliance 1200 in the user's mouth. Normal chewing can generate about 68 lbs/sq inch of pressure on the back teeth. Clenching teeth may increase that force to about 150 lbs/sq inch. This energy can be absorbed in devices such as springs 1212, 1214 and converted into electrical energy to power the appliance 1200.

In some embodiments, sensors include temperature sensors. Other sensors suitable for incorporation into monitoring system 100 include without limitation activation sensors, motion sensors, positional sensors, force sensors, optical sensors, radiation sensors, pressure sensors, atmospheric pressure sensors, pulse oximeters capnographs, airflow sensors, alcohol breathalyzers, and/or saliva sensors. Accordingly, user monitoring system 100 and related biometric data processing methods allows the multiplexing of sensors and data processing.

Activation sensors are useful for managing power requirements and power consumption of the user monitoring system 100.

Motion sensors such as gyroscopes and accelerometers are useful for measuring the motion, speed and direction of each user during a training session or game. This data can be used to measure and gauge a variety of measures including the activity level or work rate, fitness level, attentiveness, reaction time, during each game, so real-time feedback can be provided and if necessary corrections can be made and post-activity analysis can be computed. The motion sensor data can be combined with data collected from other sensors such as temperature and respiration to provide a richer biometric profile of the user and/or to alert the user who may be approaching a physical/geographic location to be avoided that includes but is not limited to dangerous, hazardous, or protected areas.

Positional sensors and/or receivers can assist the observer, e.g., a coach, a trainer, a supervisor, a manager, and/or the user, in tracking the location, the movement, acceleration, and position of the players or workers of his/her team to enhance team cohesion, protection, and effectiveness of the various specialized team members such as soccer players, American football players, military personnel or firefighters. Positional tracking of team members can also minimize, for example, the violation of the “offside” rule of soccer or field hockey or entering restricted zones. The positional sensor data can also be combined with data collected from other sensors such as temperature and respiration to provide a richer biometric profile of the user or to alert the user who may be approaching a zone to be avoided including dangerous, hazardous or protected areas.

Force sensors such as G-force sensors including accelerometers and gyroscopes are useful for detecting hits directed at the head of the user, and can also be used to compute accumulated potentially concussive hits during each game, or over multiple games. This allows the observer to relieve the user before he/she suffers cumulative brain trauma, thereby substantially increases health and safety in a manner not possible before. Such a feature is especially important in contact sports, the military and emergency personnel where hits to the head are routine such as rugby, American football, soccer, martial arts, and the like. There is medical evidence that cumulative jarring of the brain (short of a concussion that causes loss of consciousness) can, over time, cause permanent damage to the delicate brain issue, including the neurons, nerve interconnections and blood vessels. Force sensors can enable the observer to determine a player's activity level based on the amount of directional change.

Pressure sensors can detect if the user is biting down with a sufficient amount of force to ensure proper placement of dental appliance 110 thereby ensuring optimal protection of the user's jaw and teeth. One or more pressure sensors can be used to determine whether dental appliance 110 is appropriately positioned for taking accurate biometric and/or environmental data. Accordingly, data acquired before verification of proper positioning may be weighted less or discarded entirely. Pressure sensors can also aid in the rapid detection of medical issues, for example, when user loses consciousness even briefly, or experiences a seizure due to a pre-existing condition and/or due to the sporting/work activity. Pressure sensors can be used to measure grinding of teeth and clenching of the jaw to infer stress. Pressure sensors used in combination with other sensors such as respiration or airflow sensors can increase the data available to detect changes in well being. Pressure sensors can also be used to identify grinding, clenching, or other states of jaw movement.

One or more of these sensors, e.g., pressure sensor(s), optical, and/or motion (force) sensor(s), can also be used to detect usage and non-usage for the purpose of system activation and system power conservation. For example, if the dental appliance 110 has been removed, the inactivity indication can be used to trigger a low-power or sleep-mode, thereby extending the life of the power source.

Optical sensors and/or cameras can also aid to ensure the proper placement of dental appliance 110, since there should be no substantial gaps between the dental appliance and specific portions of the user's mouth, depending on the type and shape of dental appliance. Optical sensors and/or cameras can also detect if user's mouth is open, partially open, or closed. It may also possible for optical sensors and/or cameras to aid in the measurement of the user's respiration rate, especially if the user is breathing very hard through the mouth.

To ensure proper functioning of the exemplary sensors described above, especially around the pressure sensors, it is important to keep the dental appliance 110 secured within the user's mouth. Hence methods of improving adhesion to the user's mouth include adhesive microfibers, suction cups, dental adhesive, and a detachable insert for a mouth piece that attaches to the mouth piece bite plate and includes a moldable material for custom forming to the user's teeth on the upper and/or lower jaw.

In the event that the dental appliance, e.g., appliance 110, of the user is not properly placed, and to further ensure proper functioning of these sensors, an alarm indicator is embedded in the monitoring system 100 enabling the user and/or observer to be notified of such misplacement within the user's mouth or inadequate power for operation of the mouth piece.

In addition to user safety, all these exemplary sensors described above also provide important data for an analytical toolbox, enabling the user and/or observer to objectively conduct better real time and/or post-event analysis and be able to constructively critique the users' performance based on comprehensive and objective sets of data collected over a period of time. Based on the data collected, assessment, analytical and/or diagnostic strategies can be deployed that define profiles for specific users, a group of users, or the entire population of users to enhance the total system capabilities. Raw and/or processed data can also be aggregated across a broader population to create normative data including body type, nutritional conditions, general well being, and environmental conditions. Accordingly, data can reside, in whole or in part, locally on a dental appliance, e.g., appliance 110, and/or remotely on an external device, e.g., computerized storage device 840, or in a variety of locations. The data can also be transferred and shared between user monitoring system(s), e.g., system 100, observer monitoring system(s) and/or external storage device(s), device 840.

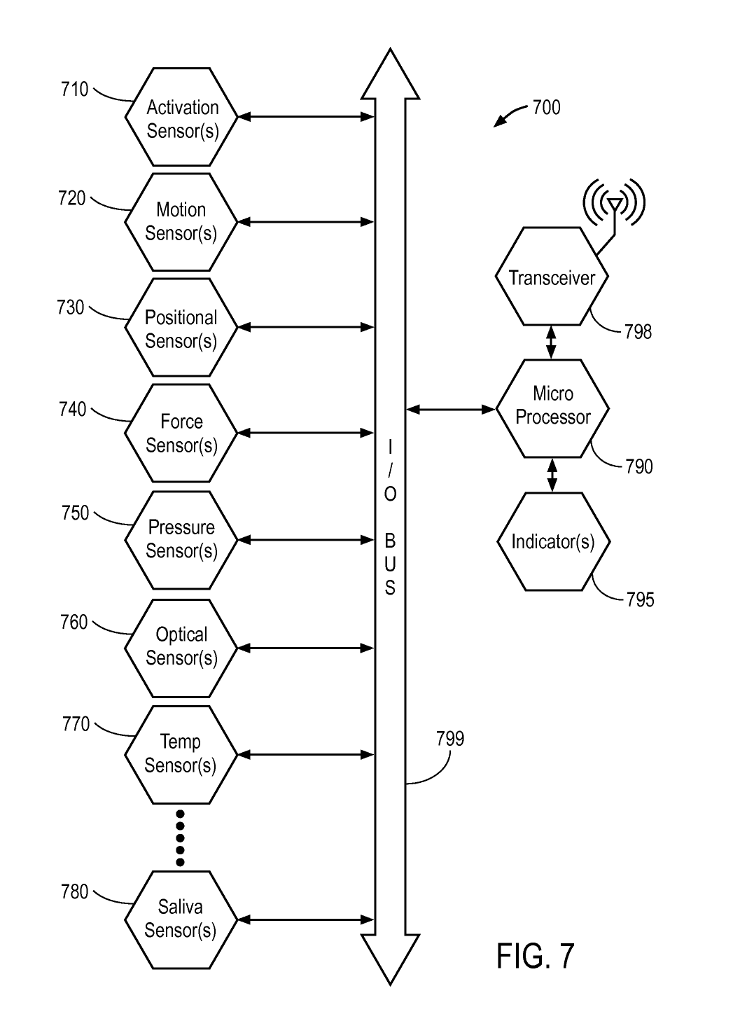

FIG. 7 is a diagram illustrating the one or more customizable functions for the user monitoring system 100 in accordance with the present invention. Based on the user's specific application of use, whether in sports, healthcare, military, or the workplace, the sensors, e.g., activation sensor 710, motion sensor 720, . . . saliva sensor 780, can incorporate diagnostic capabilities and can be used singularly by type or in combination with one or more sensors. Based on the desired configuration, the micro processor 790 can process raw data, for example, eliminate or scale invalid data point(s), and indicate when data is trending into a known area of concern for that user. Hence, computerized monitoring system 100 can be readily customized based on anticipated usage and desired data to be monitored.

In some embodiments, computerized monitoring system 100 may also includes a transmitter (not shown) coupled to processor 140, enabling system 100 to transmit data to an individual user and/or external observer monitoring system (not shown) thereby enabling the individual user and/or user's human observer to receive and analyze user vital signs, biometric data, performance data and environmental data acquired by user monitoring system 100. External observer monitoring system may be capable of receiving and processing data simultaneously from two or more user monitoring systems. External and/or user monitoring systems may also be capable of receiving and processing data from external sources, such as weather reports, normative data and historical data.

In some embodiments, the transmitter is located inside the mouth of the user and capable of wirelessly transmitting data directly to the user monitoring system 100 and/or the observer monitoring system. In other embodiments, in order to minimize the signal strength requirements of the transmitter located inside the user's mouth, monitoring system 100 also includes an optional repeater (not shown) which can be worn by the user and incorporated into their uniform, clothing, shoes, or other wearable items, e.g., worn on a belt, worn on a piece of clothing or shoe, incorporated into an ear piece, included as a part of a watch or incorporated into the helmet. The repeater is in relatively close proximity to the dental appliance 110. This repeater is configured to receive a relatively weak signal from dental appliance 110 and then relay the data at a relatively stronger signal strength to observer monitor system located some distance away, for example, from the playing field to the sidelines or an enclosed viewing booth in a stadium, which can be located up to a couple of hundred yards away. Data can also be acquired and cached by user monitoring system 100 until coupled to or within transmission range of external observer monitoring system.

In another embodiment, the transmitter is configured to be located outside the mouth of the user, such as in the user's helmet. In this configuration, the transmitter can be coupled to processor 140 via an electrical connection, an optical connection and/or a sonic connection. This connection can be incorporated into an optional tether which can provide mechanical stress and crush protection and also signal shielding.

In some embodiments, dental appliance 110 also includes one or more visual indicators (not shown) configured to be visible from outside the mouth and may indicate overall statuses. For example, a Green LED may indicate that all user parameters is within a predefined “normal” range for the user, while a Yellow LED may indicate that one or more user parameter is slightly above the “normal” range and may need attention. Conversely, a Red LED may indicate that one or more user parameter is substantially above the “normal” range and needs the immediately attention of the user and/or observer. Fiber optics may be used to reduce power consumption and provide a wider diffusion of visible light on the outward facing edge of dental appliance 110.

In addition to or in place of visual indicator(s), monitoring system 100 may include outer indicators that provide non-visual cues such as audible indicators (e.g., a speaker), tactile indicators (e.g., a vibrator) and/or taste-based indicators (e.g., a bitter, sour and/or sweet flavor). The non-visual cues can be low intensity cues intended for the user or higher intensity cues intended for the both the user and the observer.

It is also possible to provide external power (not shown) to dental appliance 110 via the tether, enabling user to use a larger external power source attached to or incorporated into the user's attire. Potential external power sources include batteries, solar cells and miniaturized fuel cells. It is also possible to recapture energy from the bite forces or head movement of the user. It is also possible to recharge these portable external power sources.

Referring to FIG. 1, air channels 112a, 112b enable the user to breath in a closed mouth position. These air channels 112a, 112b can also aid the insertion of a drinking device such as a hydration tube for the user to consume fluids from an external container such as a bottle (not shown).

Dental appliance 110 may also include one or more airflow sensors (not shown) proximate to the air channels 112a, 112b and configured to measure and send airflow directional data to processor 140, thereby enabling processor 140 to establish that dental appliance 110 is positioned correctly to collect biometric data accurately as well as to compute a respiration rate of user based on the rhythmic rate of change of airflow direction over time. Alternatively, air pressure sensors (not shown) can be incorporated into dental appliance 110 to measure cyclical changes in air pressure that can then be transposed into a respiration rate for the user. Air volume, direction and pressure data can also be used to infer lung functionality, such as capacity and efficiency. Other portable means for measuring respiration-related biometric data are also contemplated in accordance with the present invention including and not limited to, for example, measuring alcohol level using the user's breathe.

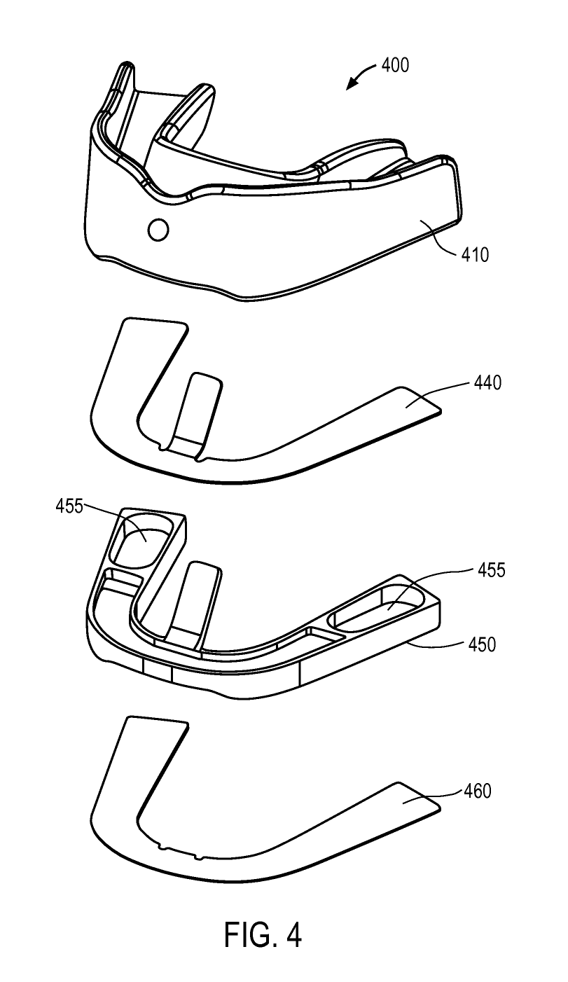

Referring now to FIG. 4, an exploded view of another embodiment of the user monitoring system 400 illustrating the sub-assemblies: dental appliance 410, processor 440, protective cover 450 and edible media 460. Dental appliance 410 is coupled to protective cover 450 which include one or more receptacles 455 configured to store and release one or more of a variety of edible and digestible substances, such as nutritional supplements, medications, antibacterial essential oils, spices and herbs, antimicrobial agents, electrolytes, vitamins, stimulants and flavors. The timing and dosage of substance release can be under manual user and/or observer control, such as by using the user's tongue to activate a pressure sensor for a calibrated dose of caffeine or Goo™ when a competitive cyclist is about to ascend a thousand vertical feet. In addition to or in place of manual control, the release schedule and dosage can also be programmed such as in accordance to a medical and/or nutritional program. In addition to a medical and/or nutritional program, this ability to store edible substances enables dental appliance 410 to inhibit the growth of bacteria and the growth of microorganisms which potentially are harmful to the user. Hence, given these examples, it is clearly contemplated that the release of substances can be for a wide variety of nutritional, medical, recreational and/or communicative purposes. Dental appliance 410 can also include a replaceable and disposable strip of media 460 which reacts to saliva in order to provide additional diagnostic assessment information on the user. This replaceable and disposable media strip 460 is a separate optional feature of monitoring system 400.

In some embodiments, the user can remove monitoring system 100 from his/her mouth and place dental appliance 110 in close proximity to a docking station (not shown) for downloading data wirelessly or via a connector. The docking system can also serve as a charging station for monitoring system 100, via for example, an electrical connector or via an inductive coupling.

In some embodiments, the user monitoring system can be implemented with rechargeable batteries in a manner that allows the user monitoring system to be placed in a storage case, or other recharging station, that includes the ability to conductively and/or inductively charge the user monitoring system. The user monitoring system storage case performs the traditional storage and protection benefits of a standard oral appliance storage case, but also has a design and power source sufficient to recharge one or more batteries and/or user monitoring system(s), as well as facilitate the storage, backup, and/or transmission of data collected by the user monitoring system(s).

The storage case can also include additional features such as UV light, high temperature steam, or other sterilization technology to clean the user monitoring system(s). The storage case can also include the appropriate sensors to extract and analyze samples of fluids and air samples collected in the user monitoring system while worn in the user's mouth. The storage case can also be used as a hub to receive wired or wireless data from the user monitoring system and/or radio frequency transmission of the data collected on the user monitoring system to an observer monitoring system or another user monitoring system or to a computer network. Docking stations can be in the form of a storage case. Docking stations can also include a multitude of docking bays to charge a plurality of monitoring systems in a team setting.

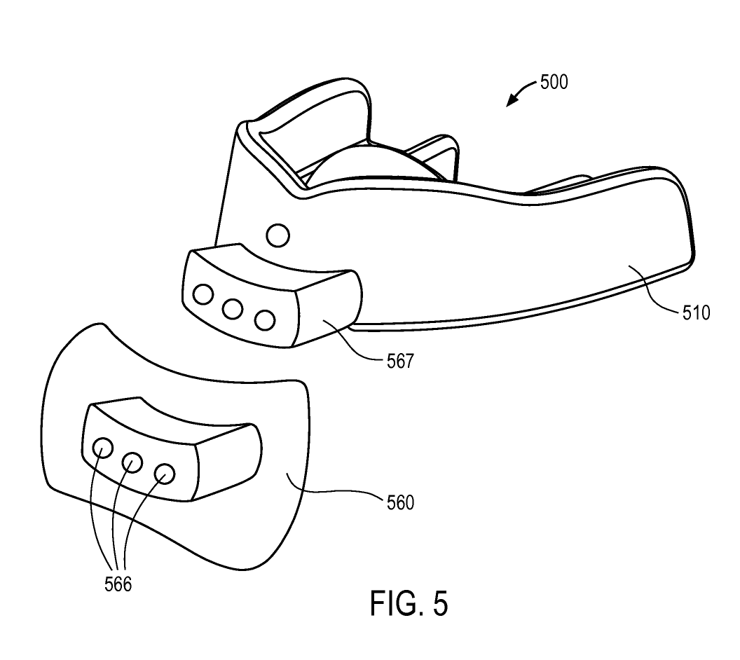

In yet another embodiment as illustrated by FIG. 5, showing user monitoring system 500, an optional lip protector 560 is operatively coupled to dental appliance 510, extending outside the user's mouth. Lip protector 560 can include one or more air channels 566 that can also facilitate the use of a drinking device such as a fluid dispensing tube or a more traditional fluid container with a “nib” that controls the fluid flow. The lip protector 560 can be secured by an attachment 567 which is coupled to, e.g., plugs or snaps into, the dental appliance 510. This attachment 567 acts as a guide for aiding the insertion and removal of dental appliance 510 to and from the user's mouth.

Lip protector 560 and attachment 567 can also house additional sensors for acquiring environmental data, such as ambient temperature, noise level, humidity and atmospheric pressure. Lip protector 560 and attachment 567 can also house a power source for monitoring system 500 and may also be detachable. In some embodiments, lip protector 560 and the attachment 567 can house processor while the sensors are housed by dental appliance 510.

Lip protector 560 can also include a variety of display technologies including lights, alpha numeric displays, graphical displays and audio displays.

In the case of the very young user and the very old user, the lip protector 560 and attachment 567 can also serve as an aid for placement and removal of dental appliance 510. An optional external key (not shown) configured to fit vents 566 lip protector 360 or configured to fit directly to dental appliance 110 can also aid placement and removal of dental appliance 110 to and from the user's mouth.

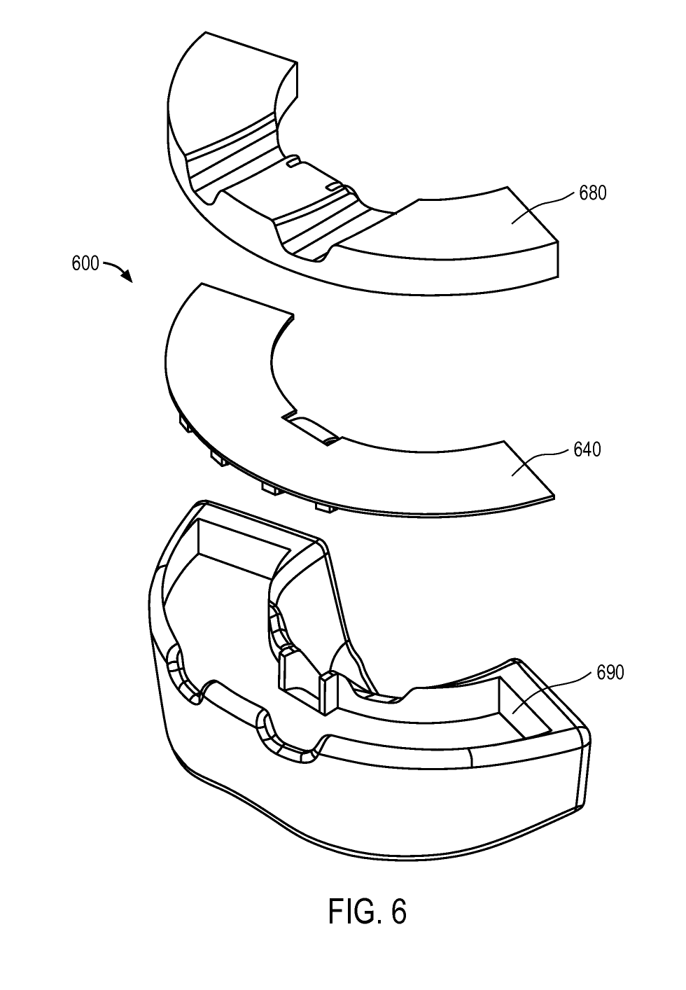

In yet another embodiment as illustrated by FIG. 6, showing user monitoring system 600 includes a dental appliance 610, configured to fit the user's lower jaw (versus the upper jaw of the user), with a recessed compartment 690 for housing a processor 640 which is shielded by a protective cover 680.

Another functional enhancement to user monitoring system(s) is interchangeability between the dental appliances fitting on the upper jaw versus the lower jaw of the user. For certain applications, it may benefit the user to have a single user monitoring system which can be worn in either position. This convertibility and reciprocate ability is contemplated by the present invention.

In some embodiments, a single user may have more than one user monitoring system depending on the nature of the activity they are performing or a preference for upper versus lower jaw placement in certain situations. Hence, a couple of user monitoring systems may be paired for a specific individual. Accordingly, multiple user monitoring systems designated for a single user may transmit data wirelessly to one or more receiving sources. Data from each of user monitoring systems designated to the single user can be aggregated or maintained separately.

It can be appreciated that a user does not need to have to have teeth for the user monitoring system to properly function. Hence, in some embodiments, the user who is missing a tooth, multiple teeth or who have removable partials or bridges may attach an optional module, similar in function to a dental bridge, in the user monitoring system's dental appliance configured to fill any of the open space(s) on their gum as they place the dental appliance in their mouth. These modules used singularly or in multiples may act as another pocket for technology components such as sensors. These modules fit in the spaces where a user does not have teeth and/or have removable dental work, and each module would be approximately 3/16″ wide with the ability to slide into place in the dental appliance wherever the user has a gap in their teeth. Whether on the upper or lower jaw of the user, the channel for the teeth on the inside of the dental appliance would create a track for the module to slide and to attach into the proper place on the dental appliance.

One or more alert devices, such as an audible alarm, can also be incorporated in user monitoring system 100 and/or observer monitoring system. Alerts can be triggered under a wide variety of manual and programmed conditions, such as battery-low-power, data storage overflow warning, insertion of dental appliance 110 prior to competition and removal of appliance 110 thereafter, and whenever acquisition of biometric data and/or environmental data is needed.

Many other functional enhancements to monitoring system 100 and/or observer monitor are also possible. For example, monitoring system 100 can include a transceiver capable of two-way communication instead of a transmitter. Blood oxygenation data may also be collected by monitoring system 100 using optical or other means such as a pulse oximeter or capnograph. Other exemplary enhancements include incorporating microphone(s) and/or speaker(s) thereby enabling the observer and the user to communicate with each other. Microphones can be used in combination with signal processing occurring locally on the user monitoring system or remotely by another processing unit. The data collected by the microphone(s) can be processed to identify the various sounds, categorize the sounds, and/or recognize the events (e.g., speech, snoring, respiration, choking and external noises such as explosion) to infer situational conditions. These categorized sounds can be transmitted to the user and/or other observers. GPS and/or RFID receivers can also be incorporated in monitoring system 100 to provide geographical positional data, enabling a supervisor to track, for example, a team of human fruit pickers.

Many alternate form factors are also contemplated for user monitoring system 100 and observer monitoring system. For example, the observer monitoring system can be implemented as an application for smart phones, e-books, heart rate monitors, bicycle computers or tablet PCs. The novel concepts of the present invention described above may also be applicable to military personal equipment, workplace protective equipment, and medical devices. In applications where the users are geographically dispersed and/or where transmission obstacles exist (e.g., by a steep canyon wall), networking concepts known to one skilled in the art such as data hopping can also be implemented whereby an intermediate user may relay data from a remote user back to the observer, i.e., function as a repeater.

With this wealth of user biometric data and environmental data being transmitted by one or more user monitoring systems to the computerized observer monitoring system, many avenues and strategies for analysis and heuristically processes are now possible, including statistical and/or analytical techniques known to one skilled in the data processing arts. Data collected from a wide variety of different sensors can also be used to objectively correlate and cross-validate data, thereby increasing the reliability and accuracy of the data collected. Such processing workload can either be assigned to one or more of user monitoring system 100, the observer monitoring system, and a remote processing system. As discussed above, FIG. 8 illustrates the user monitoring system(s) 811, 812, . . . 819 and observer monitoring system 820 collecting and sharing biometric and environmental data via a two-way means of communication. Such an embodiment 800 allows real-time data collection, sharing and aggregation.

For example, temperature sensors can be used in a variety of creative ways. For example, “normal” user temperature profiles can be developed for individual users during active and relative inactive periods. These “normal” profiles can serve as an early warning system that enables the observer to pay attention to the user before significant deterioration of the user's health or performance occurs. Profiles can also be developed based on other sensors types. Combinational profiles can also be developed based on multiple sensor types. It is also possible to further adapt these user profiles to accommodate many different environmental factors and also clothing worn by the user. Examples of factors and clothing include climate, ambient temperature, humidity, chemicals present in the air, changes in air pressure, elevation of the geographical location and clothing apparel and accessories such as insulated helmets, shoes, and other protective gear.

Once placed in the mouth and positioned properly, presets can be applied for each user based on normative data within a specific user and/or across the user population. The user monitoring system can use the presets as the beginning data point in calculating the data trending and data averaging methods benefitting the user and can be used for other functions including defining custom notification thresholds.

Caloric burn rate can be inferred from such temperature readings over time. Temperature readings can also be used to as a guide to hydration strategies, to minimize the occurrence of extreme heat related conditions such as heat exhaustion and heat strokes. Conversely, temperature readings can also be used as a guide to rest periods in a heated area to minimize the occurrence of extreme cold conditions such as frost bites.

The user monitoring system in accordance with the present invention can assess when a user's temperature rises substantially close to or over 100 degrees Fahrenheit which further promotes safety for that user and/or observer. Accordingly, a fever resulting in an elevated temperature can be the early warning sign of an infection that may need medication to address the infection. This data becomes very important in the consistent monitoring of cancer patients where their immune system is already compromised by cancer treatments such as chemotherapy, radiation and immunotherapy. Further, a body temperature above 100 degrees Fahrenheit may also be a sign of a concussion, and once a concussive event is suspected, an elevated temperature is a condition that is watched for in the user and/or observer. Further, measuring body temperature over time can be used to track the ovulation cycle of females.

Sleep disturbances can also affect a user's temperatures and the user monitoring system can help monitor temperature fluctuations during sleep that may be caused by sleep apnea, insomnia, and other sleep disorders.

To improve the efficiency and accuracy of data analysis, it is also possible to preload processor 140 and/or individual observer monitor system and/or observer monitor system with initial preset limits and profiles for generic users, individualized athlete profiles and for specific activities. Preloading can be very advantageous since specific activities and specific team-roles can impose very different demands on the user. For example, a mixed martial artist has very different demands from that of a speed skater, and a soccer goal keeper's activities are very different from that of a defender. Similarly, an American football quarterback's physical and mental demands are very different from that of an offensive lineman.

To further improve the efficiency and accuracy of data analysis, it is also possible to correlate and to factor the variations in temperature that result in circadian rhythm, time of day, and other circumstances affecting a user's temperature.

By comparing data from multiple sensors of the same type, and/or across sensors of different types, it is also possible to cleanse data by detecting and partially or fully omitting aberrant data from, for example, malfunctioning or misaligned sensors. Data cleansing can be implemented in real-time or during post-activity analysis. Data cleansing can also applied across multiple data sets acquired during several separate activities.

To further protect the user monitoring system electronics which gather and process data, a potting material can be wrapped around the microprocessor and related components to provide a method of encapsulation of these components. This encapsulation further protects the user's tissue and tongue while the dental appliance is worn. This creates a technology capsule that can be placed in the dental appliance. The potting material can also enhance the ability of the electronics to withstand the high temperatures inherent in the injection molding process for manufacturing dental appliances.

In yet another embodiment of this present invention, an independent external module can be attached to an existing user's dental appliance such as a mouth guard that an individual already owns. Hence the ability to adapt “after-market” technology module that can be attached to an existing oral appliance through multiple ways consistent with the form factor of the existing oral appliance. Methods of attachment include rubber posts, adhesive and technology module adapter which allow the supplemental technology module to enhance the functionality of an existing user monitoring system. This flexibility also enables independent modules to be reused or repurposed as accessories between different dental appliances.

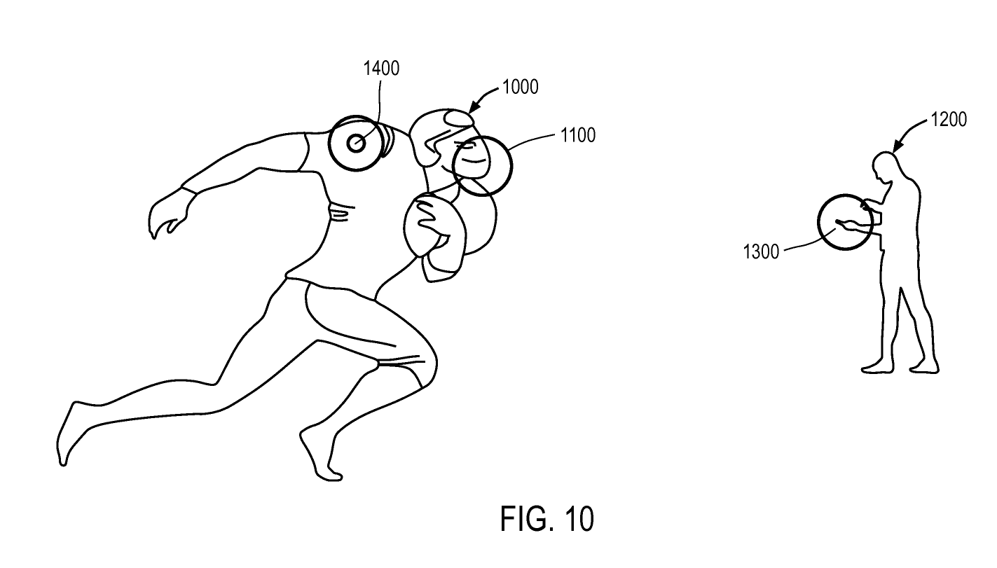

FIG. 10 illustrates an exemplary embodiment applicable to an athlete 1000 and related observer 1200. With dental appliance 1100 worn by athlete 1000, observer 1200 can monitor biometric data gathered by dental appliance 1100 and transmitted to receiver 1300. To minimize the signal strength requirements of the transmitter located inside the athlete's mouth, the monitoring system can include a repeater 1400.

In some embodiments, the user monitoring system deploys an extension of the temperature algorithm to recognize the operational effectiveness of the user body's ability to dissipate heat, thereby using the core body temperature to determine if the user is recovering from an unusual rise in core body temperature. There are generally four primary ways the user's body can dissipate heat: conduction (i.e., skin touching something cooler), convection (i.e., cooler air currents touching the body), radiation (i.e., radiating electromagnetic waves leaving the user's body), and evaporation (i.e., sweating). Radiation accounts for 50-65% of heat loss and evaporation accounts for 30-35% of heat loss. Radiation becomes ineffective once the user's body's core temperature reaches 35 degrees Celsius, and evaporation ceases to be effective at 100% humidity (body covered in sweat). Thus, there are times when human activity will cause the body's core temperature to rise, and the body's systems are unable to dissipate the heat. Thus, in addition to calculating the core body temperature, monitoring the core body temperature and knowing when the body is no longer recovering, it indicates the individual is in danger besides the absolute temperature number. For example, a user may no longer be dissipating heat when their core body temperature is 39 degrees Celsius (slightly above normal), but the user needs to cease their activity or they will enter heat exhaustion and potentially heat stroke. Thus, the user monitoring system is able to measure the effects of normal core body temperature fluctuations of the user (i.e., “continuously calculated temperature variations”) and recognize when the user is not fluctuating on their “normal” basis.

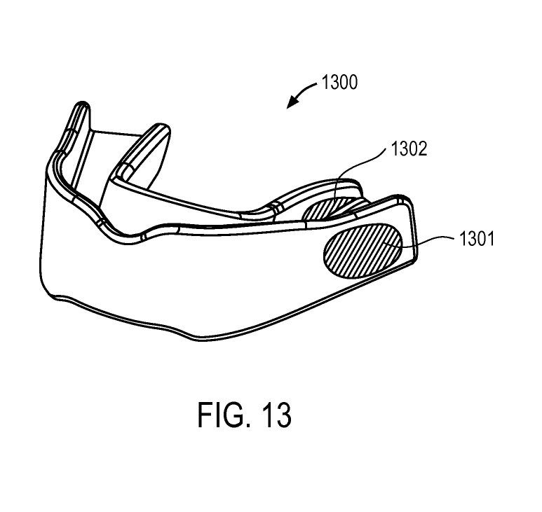

In another embodiment, the dental appliance can include pulse oximeter technology as illustration in FIG. 13. As shown in FIG. 13, the pulse oximeter 1300 consists of a light source 1301 and a photodetector 1302 which reads through the vascular bed of a user's gum. Pulse oximeter technology can be used to enhance performance and protection by providing a measurement of a variety of conditions including the oxygen saturation in the blood and changes in blood volume, assist in detection of when ventilation is inadequate to perform necessary gas exchange in the lungs (hypoventilation), estimate heart rate by measuring cyclic changes in light transmission, and is a biomarker for diagnosis of sleep apnea.

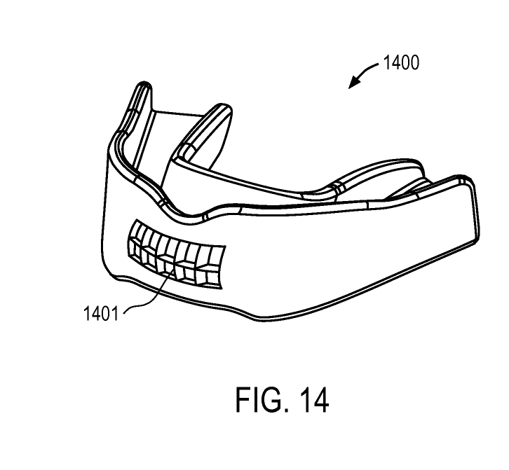

In another embodiment, the dental appliance can include capnograph technology as illustrated in FIG. 14. As shown in FIG. 14, the non-invasive capnograph technology 1400 can be used to enhance performance and protection by providing a monitoring of a variety of variables including the amount of carbon dioxide in the expired air. The capnograph sensor 1401 monitors CO2 production as well as respiratory patterns. Too much carbon dioxide would suggest that the gas exchange in the lungs is not functioning.

In another embodiment, receptacles built into the dental appliance can also capture exhaled air. This captured air can be processed in real time or post event using mass spectrometry to identify the conditions of the user's body. Monitoring and analyzing expelled air during breathing with a dental appliance can be used for a variety of biometric monitoring including detection of problems of oxygen and carbon dioxide exchange in the lungs, the presence of drugs such as alcohol, and other biomarkers such as aldehydes which indicate a variety of circumstances including oxidative stress due to excessive exercise.

The receptacles could be in a variety for forms including a channel with collection area within the dental appliance, a closed receptacle that is operated manually by user biting pressure on the dental appliance that mechanically opens the receptacle, by electromechanical means initiated by the user at a preset time or as initiated by a remote communication received through a transceiver located on the dental appliance. The channel and/or receptacle can also include a membrane or absorbent material that collects the samples. Processing of the samples can occur on the dental appliance and/or remotely.

As shown in FIG. 4, uses of the receptacles include salivary sampling and assessment of a user's pH and their saliva viscosity. This aids in monitoring a user's hydration level/status and provides a diagnostic tool for potential early detection of health related problems associated with a wide array of human conditions including; but not limited to, dehydration, poor dental care, unhealthy balance between acid and alkaline seen through a user's pH, poor digestive enzyme activity, harmful bacteria detection, indication of recent illegal drug use and identification of medical allergies.

While this invention has been described in terms of several embodiments, there are alterations, modifications, permutations, and substitute equivalents, which fall within the scope of this invention. Although sub-section titles have been provided to aid in the description of the invention, these titles are merely illustrative and are not intended to limit the scope of the present invention.

It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A computerized monitoring system for monitoring data of a user, the monitoring system comprising:
a dental appliance configured to fit substantially inside a mouth of a user;
at least one temperature sensor incorporated into the dental appliance, and wherein the at least one temperature sensor is configured to monitor temperature data of the user;
at least one pressure sensor incorporated into the dental appliance, and wherein the at least one pressure sensor is configured to measure pressure exerted by the user; and
a processor incorporated into the dental appliance, wherein the processor is coupled to the at least one temperature sensor and to the at least one pressure sensor, wherein the processor is configured to acquire and process temperature data from the at least one temperature sensor, and wherein the processor is further configured to acquire and process pressure data from the at least one pressure sensor.

2. The monitoring system of claim 1 further comprising an indicator coupled to the processor, wherein the indicator is configured to indicate at least one of a temperature indication and a pressure indication from the processor.

3. The monitoring system of claim 1 further comprising a transmitter coupled to the processor.

4. The monitoring system of claim 1 further comprising a transceiver coupled to the processor.

5. The monitoring system of claim 3 wherein the transmitter is incorporated into the dental appliance, and wherein the transmitter is configured to transmit data from the processor wirelessly to an external observer monitoring system.

6. The monitoring system of claim 3 wherein the transmitter is incorporated into the dental appliance, wherein the transmitter is a short range transmitter configured to transmit data from the processor to a repeater located in close proximity to the transmitter, and wherein the repeater is configured to relay data from the transmitter to an external receiver.

7. The monitoring system of claim 6 wherein the repeater is incorporated into or attached to an article of clothing worn by the user.

8. The monitoring system of claim 7 wherein the article of clothing is one of a helmet, a footwear and a wearable electronic device.

9. The monitoring system of claim 3 wherein the transmitter is located outside the mouth of the user, wherein the transmitter is coupled to the processor via a physical connection, and wherein the transmitter is configured to transmit data from the processor wirelessly to an external observer monitoring system.

10. The monitoring system of claim 9 wherein the physical connection includes at least one of an electrical connection, an optical connection, and/or a sonic connection.

11. The monitoring system of claim 9 wherein the physical connection is incorporated into a tether coupling the dental appliance to an article of clothing worn by the user.

12. The monitoring system of claim 11 wherein the article of clothing is a helmet.

13. The monitoring system of claim 11 wherein the tether includes a power connection between the dental appliance and an external power source.

14. The monitoring system of claim 13 wherein the external power source is at least one of a solar power source, a battery, and a fuel cell.

15. The monitoring system of claim 1 further comprising an airflow sensor incorporated into the dental appliance and configured to measure and send airflow directional data to the processor, thereby enabling the processor to compute a respiration rate of the user.

16. The monitoring system of claim 1 further comprising an air-pressure sensor incorporated into the dental appliance and configured to measure and send air-pressure data to the processor, thereby enabling the processor to compute a respiration rate of the user.

17. The monitoring system of claim 1 wherein the dental appliance also includes an extension configured to extend outside the mouth of the user.

18. The monitoring system of claim 1 wherein the extension is a lip protector configured to protect lips and teeth of the user.

19. The monitoring system of claim 17 wherein the extension configured to couple to a power source.

20. The monitoring system of claim 17 wherein the extension includes a temperature sensor for measuring ambient temperature.

21. The monitoring system of claim 1 further comprising a microphone and an audio transducer configured to facilitate audio communications between the user and at least one of another user and an observer.

22. The monitoring system of claim 1 wherein the at least one pressure sensor is activated by a tongue of the user and contact with at least one of an upper palette, a lower palette and teeth of the user.

23. The monitoring system of claim 1 wherein the processor is coupled to a power storage device incorporated into the dental appliance and configured to accept an electrical charge from an external charger.

24. The monitoring system of claim 1 wherein the processor is coupled to an input-output device incorporated into the dental appliance and configured to dock to an external observer monitor for downloading and uploading of data to and from the processor.

25. The monitoring system of claim 1 further comprising a hydration sensor incorporated into the dental appliance and configured to sense a hydration level of the user.

26. The monitoring system of claim 1 further comprising a positional sensor incorporated into the dental appliance and configured to sense a geographical position of the user.

27. The monitoring system of claim 1 further comprising a receptacle configured to store and release an edible substance.

28. The monitoring system of claim 1 wherein the processor is further configured to cleanse the temperature data and the pressure data.

29. The monitoring system of claim 1 wherein the at least one pressure sensor is activated by the dental appliance contacting with at least one of an upper palette, a lower palette and teeth of the user.

30. The monitoring system of claim 1 further comprising a carrying case configured to store at least one dental appliance, and wherein the carrying case is coupled to a power source to accept an electrical charge from an external charger for the monitoring system.

31. The monitoring system of claim 1 further comprising a radiation sensor configured to measure ambient radiation level.

32. The monitoring system of claim 4 wherein the data communication is bidirectional between the dental appliance and an external observer monitoring system.

33. The monitoring system of claim 1 further comprising at least one biological hazard detector.

34. The monitoring system of claim 1 further comprising at least one chemical hazard detector.

35. The monitoring system of claim 1 further comprising at least one force sensor incorporated into the dental appliance and wherein the at least one force sensor is configured to measure force experienced by the user.

36. The monitoring system of claim 1 further comprising a pulse oximeter coupled to the processor, wherein the processor is further configured to compute oxygen saturation in the user's blood.

37. The monitoring system of claim 1 further comprising a capnometer coupled to the processor, wherein the processor is further configured to compute the amount of carbon dioxide in the user's breath.

38. The monitoring system of claim 1 further comprising at least one exhaled air sensor configured to acquire and analyze an air sample of the user.

39. The monitoring system of claim 1 further comprising at least one receptacle to capture a sample of at least one of saliva and air from the user.

40. The monitoring system of claim 39 wherein the processor is further configured to associate a time stamp with the sample from the user.

41. The monitoring system of claim 1 further comprising a kinetic energizer configured to utilize kinetic energy from the compression of the user's bite.

42. The monitoring system of claim 1 further comprising an inductive energizer.

43. The monitoring system of claim 4 further comprising the capability to wirelessly send and receive data and notifications with a second monitoring system.

44. The monitoring system of claim 17 wherein the extension includes an atmospheric pressure sensor for measuring ambient air pressure.

45. The monitoring system of claim 1 further comprising an optical sensor configured to measure light levels.

46. A computerized method for monitoring data of a user using a dental appliance configured to fit substantially inside a mouth of the user, the monitoring method comprising:
measuring temperature data of a user using at least one temperature sensor incorporated into the dental appliance, the dental appliance configured to fit substantially inside a mouth of the user;
measuring pressure exerted by the user using at least one pressure sensor incorporated into the dental appliance; and
acquiring the temperature data from the at least one temperature sensor and processing the acquired temperature data using a processor incorporated into the dental appliance; and
acquiring the pressure data from the at least one pressure sensor and processing the acquired pressure data using the processor of the dental appliance.

47. The method of claim 46 further comprising providing at least one of a temperature indication and a pressure indication from the processor.

48. The method of claim 46 further comprising transmitting data from the monitoring system.

49. The method of claim 48 further comprising receiving data for the monitoring system.

50. The method of claim 46 further comprising computing oxygen saturation in the user's blood.

51. The method of claim 46 wherein the at least one pressure sensor is activated by the dental appliance contacting with at least one of an upper palette, a lower palette and teeth of the user.

52. The method of claim 46 wherein the data communication is bidirectional between the dental appliance and an external observer monitoring system.

53. The method of claim 46 further comprising measuring ambient air temperature.

54. The method of claim 46 further comprising measuring light levels.

55. The method of claim 46 further comprising computing a respiration rate of the user.

56. A computerized monitoring system for monitoring data of a user and providing the user data to an observer, the monitoring system comprising:
at least one user monitoring system having:
a dental appliance configured to fit substantially inside a mouth of a user;
at least one temperature sensor incorporated into the dental appliance, and wherein the at least one temperature sensor is configured to monitor temperature data of the user;
at least one pressure sensor incorporated into the dental appliance, and wherein the at least one pressure sensor is configured to measure pressure exerted by the user;
a processor incorporated into the dental appliance, wherein the processor is coupled to the at least one temperature sensor and to the at least one pressure sensor, wherein the processor is further configured to acquire and process temperature data from the at least one temperature sensor, and wherein the processor is further configured to acquire and process pressure data from the at least one pressure sensor; and
a transmitter configured to transmitting the processed temperature data and the processed pressure data from the processor; and
at least one observer monitoring system having:
a receiver configured to receive the processed temperature data and the processed pressure data from the transmitter of the user monitoring system; and
an interface configured to provide the processed temperature data and the processed pressure data from the receiver to an observer.
